(19) United States
(12) Patent Application Publication
(10) Pub. No.: US 2022/0370397 A1
(43) Pub. Date: Nov. 24, 2022

(54) METHODS AND COMPOSITIONS FOR TREATMENT OF CANCER

(71) Applicant: Enzychem Lifesciences Corporation, Seoul (KR)

(72) Inventors: Ki-Young Sohn, Seoul (KR); Jae Wha Kim, Daejeon (KR); Sun Young Yoon, Daejeon (KR)

(21) Appl. No.: 17/770,990

(22) PCT Filed: Oct. 28, 2020
(86) PCT No.: PCT/IB2020/060104

§ 371 Date: Apr. 21, 2022

Related U.S. Application Data
(60) Provisional application No. 62/926,963, filed on Oct. 28, 2019.

(51) Int. Cl. A61K 31/231; A61K 31/704; A61K 31/675; A61P 35/00

(52) U.S. Cl. CPC A61K 31/231; A61K 31/704; A61K 31/675; A61P 35/00

(57) ABSTRACT

In one aspect, methods and compositions are provided for treating a neoplasia such as a solid tumor, the methods and composition comprising a) one or more chemotherapeutic agents such as a checkpoint inhibitor or doxorubicin and/or cyclophosamide; and b) a monoacetyl diacylglycerol compound such as 1-palmitoyl-2-linoleoyl-3-acetylglycerol (PLAG)

### FIELD

In one aspect, methods and compositions are provided for treating a neoplasia such as a tumor, the methods and compositions comprising a) one or more chemotherapeutic agents such as AC-regimen and b) a monoacetyl diacylglycerol compound such as 1-palmitoyl-2-linoleoyl-3-acetylglycerol (PLAG).

### BACKGROUND

Cancers are characterized by abnormal and uncontrolled cell growth. Cancer can involve any tissue in the body, and can spread outside the tissue of origin. Uncontrolled proliferation and other cellular abnormalities can lead to the formation of cancerous tumors. Tumors can disrupt the function of and destroy the tissues in which they originate, and, when cancer cells metastasize, secondary tumors can develop near to or disparate from the site of primary growth. Causes of cancer have been linked to various chemicals, viruses, bacteria, and environmental exposures.

It thus would be desirable to have improved cancer therapies.

### SUMMARY

In one aspect, we now provide new therapies for treatment and prevention of a patient suffering from cancer.

In one aspect, methods and compositions are provided for reducing or suppressing tumor growth in a patient, that includes administration of one or more chemotherapeutic agents in conjunction with administration of a monoacetyl diacylglycerol compound of Formula (I), where the compound of Formula (I) is distinct from the one or more chemotherapeutic agents.

We surprisingly found that coordinated administration of monoacetyl diacylglycerol compound of Formula (I) and one or more chemotherapeutic agents can produce synergistic effects in decreasing tumor size/burden. See, for instance, the results of Example 1 and 3 which follows.

In one aspect, the present methods comprise administering to a subject such as a human having a tumor or other neoplasia a therapeutically effective amount of:

a) one or more chemotherapeutic agents; and

b) a monoacetyl diacylglycerol compound of Formula (I):

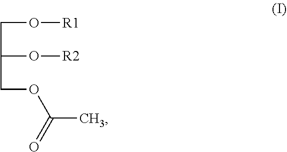

wherein R1 and R2 are independently a fatty acid group comprising 14 to 20 carbon atoms. The b) compound of Formula (I) is distinct from the a) one or more chemotherapeutic agents. The a) one or more chemotherapeutic agents and b) compound of Formula (I) are suitably administered to the patient in combination or other coordinated manner.

In preferred aspects, the b) monoacetyl diacylglycerol is a compound of Formula II:

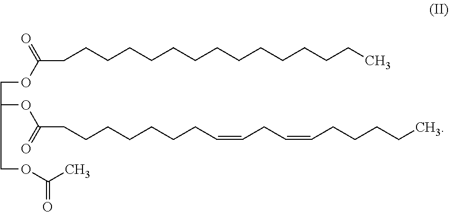 [SMILES: CCCCC/C=C\C/C=C\CCCCCCCC(=O)OC(COC(C)=O)COC(=O)CCCCCCCCCCCCCCC]

The compound of Formula (II) is also referred to herein as PLAG (1-palmitoyl-2-linoleoyl-3-acetylglycerol), PLAG or EC-18.

In preferred aspects, the one or more chemotherapeutic agents in addition to PLAG comprise one or more immune checkpoint inhibitor compounds or agents. In some embodiments, the immune checkpoint inhibitor is an antibody or fragment thereof, including a monoclonal antibody or fragment thereof.

Preferred checkpoint inhibitor agents for use in the present compositions and methods include PD-1 inhibitors such as Pembrolizumab (Keytruda), Nivolumab (Opdivo); and Cemiplimab (Libtayo). Additonal preferred checkpoint inhibitor agents for use in the present compositions and methods include PD-L1 inhibitors such as Atezolizumab (Tecentriq); Avelumab (Bavencio); and Durvalumab (Imfinzi). Additonal preferred checkpoint inhibitor agents for use in the present compositions and methods include CTLA-4 inhibitors such as Ipilmumab (Yervoy).

In certain preferred aspects, the checkpoint inhibitor is a PD-L1 inhibitor.

We surprisingly found that coordinated administration of monoacetyl diacylglycerol compound of Formula (I) and one or more checkpoint inhibitor agents can produce synergistic effects in cancer treatment effects, including decreased tumor size/burden. See Example 3 which follows.

In certain preferred aspects, the one or more chemotherapeutic agents comprise doxorubicin.

In additional preferred aspecyts, trhe one or more chemotherpaueitc agents comprise cyclophosphamide.

In certain aspects, the one or more chemotherapeutic agents comprise both doxorubicin and cyclophosphamide, where those agents may be administered at least substantially simultaneously or sequentially.

In additional preferred aspects, the one or more chemotherapeutic agents comprise 5-FU (5-fluorouracil) and/or cisplatin.

In particular preferred aspects, the one or more chemotherapeutic agents comprise AC regimen which includes doxorubicin such as doxorubicin hydrochloride (Adriamycin) and cyclophosphamide.

In particular preferred aspects, the one or more chemotherapeutic agents comprise AC-T or AC-Taxol regimen which includes doxorubicin such as doxorubicin hydrochloride (Adriamycin) and cyclophosphamide, followed by treatment with paclitaxel (Taxol).

Other chemotherapeutic agents that may be administered to a subject in accordance with the present methods include for instance cyclophosphamide etoposide, ifosfamide, mesna, gemcitabine and/or tamoxifen, or one or more other chemotherapeutic agents.

In certain embodiments, the treatment approaches of the invention also may also be combined with any of the following therapies: radiation, chemotherapy, surgery, therapeutic antibodies, immunomodulatory agents, proteasome inhibitors, pan-DAC inhibitors, H-DAC inhibitors, checkpoint inhibitors, adoptive cell therapies include CAR T and NK cell therapy and vaccines.

In certain aspects of the invention, the a) one or more chemotherapeutic agents do not include granulocyte-colony stimulating factor (G-CSF). In certain aspects, a subject is not administered granulocyte-colony stimulating factor (G-CSF) as part of or in conjuniction with the methods, compositions or kits disclosed herein. In certain aspects, a subject has not been administered granulocyte-colony stimulating factor (G-CSF) for at least 0.5, 1, 2, 3, 4, 6, 8 weeks or more before being treated with the a) one or more chemotherapeutic agents and b) compound of Formula (I) as dislosed herein, and/or a subject has not been administered granulocyte-colony stimulating factor (G-CSF) for at least 0.5, 1, 2, 3, 4, 6, 8 weeks or more after being treated with the a) one or more chemotherapeutic agents and b) compound of Formula (I) as dislosed herein.

In a further aspect, pharmaceutical compositions are provided comprising a) one or more chemotherapeutic agents such as doxorubicin and/or one or more checkpoint inhibitor compounds; and b) a monoacetyl diacylglycerol compound such as PLAG (1-palmitoyl-2-linoleoyl-3-acetylglycerol) that is distinct form the a) one or more chemotherapeutic agents.

In a yet further aspect, kits are provided for use to treat or prevent a neoplasia including a solid tumor. Kits of the invention suitably may comprise a) one or more chemotherapeutic agents; and b) a monoacetyl diacylglycerol compound such as PLAG (1-palmitoyl-2-linoleoyl-3-acetylglycerol) that is distinct from the a) one or more chemotherapeutic agents. Preferably, a kit will comprise a therapeutically effective amount of each of the a) one or more chemotherapeutic agents and b) monoacetyl diacylglycerol compound such as PLAG. Preferred kits also may comprise instructions for use of the a) one or more chemotherapeutic agents and b) monoacetyl diacylglycerol compound such as PLAG to treat a neoplasia such as a solid tumor, including breast cancer. The instructions suitably may be in written form, including as a product label.

Methods, compositions and kits of the invention may be used to treat subjects suffering from a variety of types of neoplasias and cancers. In certain aspects, the methods, compositions and kits may be used to treat a subject suffering from breast cancer.

In certain aspects, methods of the invention include identifying and selecting a subject suffering from a neoplasia, such as a solid tumor, or breast cancer, and then administering to the selected subject the a) one or more chemotherapeutic agents and b) monoacetyl diacylglycerol compound such as PLAG.

As discussed, in certain aspects, compositions and kits dislosed herein do not include granulocyte-colony stimulating factor (G-CSF).

Other aspects of the invention are disclosed infra.

### BRIEF DESCRIPTION OF DRAWING

The patent or application file contains at least one drawing executed in color. Copies of this patent or patent application publication with color drawing(s) will be provided by the Office upon request and payment of the necessary fee.

FIG. 1 shows an exemplary experimental design to investigate the synergistic effect on therapeutic efficacy of tumor using simultaneous treatment of PLAG and AC-regimen.

FIGS. 2A-2J show synergetic effects of co-administrating PLAG and AC regimen. Compared to positive control: *P<0.05, ***P<0.001 (each experiment n=5). Compared to regimen treated group: #P<0.05, ##P<0.01 (each experiment n=5). N.S., not significant)

FIG. 2A shows the change of weekly recorded tumor mass in Xenograft mouse during AC-regimen treatment (2/20) and PLAG co-treatment.

FIG. 2B shows analysis of tumor weight of each group from FIG. 2A measured at sacrifice day.

FIG. 2C shows tumor mass in xenograft mice during AC-regimen (2/20) and PLAG co-treatment shown in FIG. 2A.

FIG. 2D shows staining and apoptosis-related protein expression of tumor tissue in the sacrificed mice after AC-regimen treatment (2/20) and PLAG co-treatment of FIG. 2A.

FIG. 2E shows the changes in apoptosis-related protein expression level in tumor tissue at sacrifice after AC-regimen (2/20) and PLAG co-treatment shown in FIG. 2A.

FIG. 2F shows the change of weekly recorded tumor mass in Xenograft mouse during AC-regimen treatment (5/50) and PLAG co-treatment.

FIG. 2G shows analysis of tumor weight of each group from FIG. 2F measured at sacrifice day.

FIG. 2H shows tumor mass in xenograft mice during AC-regimen (5/50) and PLAG co-treatment shown in FIG. 2F.

FIG. 2I shows staining and apoptosis-related protein expression of tumor tissue in the sacrificed mice after AC-regimen treatment (5/50) and PLAG co-treatment of FIG. 2F.

FIG. 2J shows the changes in apoptosis-related protein expression level in tumor tissue at sacrifice after AC-regimen (5/50) and PLAG co-treatment shown in FIG. 2F. ##P<0.01 (each experiment n=5). N.S., not significant.

FIGS. 3A-3D show suppression or inhibition of neutrophil chemotaxis during co-administrating PLAG and AC-regimen.

FIG. 3A shows verification of tumor infiltration neutrophil using immunohistochemistry with antibodies. Anti-Ly6C+/Ly6G+ antibodies and anti-Ly6G+ only antibodies were used.

FIG. 3B shows evaluation of tumor infiltrating neutrophil. Compared to negative control: *P<0.05, **P<0.01, ***P<0.001 (each experiment n=5). N.S., not significant. Compared to AC-regimen group: #P<0.05, ##P<0.01, ###P<0.001 (each experiment n=5). N.S.′, not significant.

FIG. 3C shows evaluation of secreting chemokines associated with neutrophil chemo-taxis in blood: *P<0.05, **P<0.01, ***P<0.001 (each experiment n=5). N.S., not significant. Compared to AC-regimen control group: #P<0.05, ##P<0.01, ###P<0.001 (each experiment n=5). N.S.′, not significant.

FIG. 3D shows evaluation of neutrophil in blood: *P<0.05, **P<0.01, ***P<0.001 (each experiment n=5). N.S., not significant. Compared to AC-regimen control group: #P<0.05, ##P<0.01, ###P<0.001 (each experiment n=5). N.S.′, not significant.

FIGS. 4A-4G show growth inhibition of cancer by PLAG treatment.

FIG. 4A shows effects of PLAG on tumor mass in Xenograft mice.

FIG. 4B shows verification of tumor weight in Xenograft mice on sacrifice day. Compared to positive control: *P<0.05, ***P<0.001 (each experiment n=5).

FIG. 4C shows change of weekly counted tumor mass in PLAG treated Xenograft mice. FIG. 4D shows verification of tumor infiltration neutrophil using immunohistochemical. Compared to positive control: *P<0.05, **P<0.01 (each experiment n=5).

FIG. 4E shows evaluation of secreting chemokines associated with neutrophil chemo-taxis in blood: compared to negative control; *P<0.05, **P<0.01, ***P<0.001 (each experiment n=5). N.S., not significant. Compared to positive control; #P<0.05, ##P<0.01, ###P<0.001 (each experiment n=5). N.S.′, not significant.

FIG. 4F shows verification of neutrophil chemotaxis related chemokine expression in tumor using immunohistochemistry.

FIG. 4G shows verification of tumor cell cycle inducing factor expression in tumor using immunohistochemistry.

FIGS. 5A-5D show inhibition of cancer growth and neutrophil infiltration by PLAG treatment.

FIG. 5A shows PLAG effects on tumor mass in Xenograft mice.

FIG. 5B shows verification of Tumor weight in Xenograft mice on sacrifice day. Compared to positive control: *P<0.05, **P<0.01 (each experiment n=5). N.S., not significant.

FIG. 5C shows change of weekly counted tumor mass in PLAG treated Xenograft mice.

FIG. 5D shows Verification of tumor infiltration neutrophil using immunohistochemistry.

FIGS. 6A-6B show inhibition of cancer proliferation by enhanced PAR2 degradation.

FIG. 6A shows inhibition of cell growth by PLAG treatment with dose-dependent manner in neutrophil-activated MDA-MB-231 breast cancer cells. Compared to negative control each week: *P<0.05, **P<0.01, ***P<0.001 (each experiment n=5). N.S., not significant. Compared to neutrophil stimulated only group each week: #P<0.05, ##P<0.01, ###P<0.001 (each experiment n=5). N.S.′, not significant.

FIG. 6B shows inhibitory effect of PLAG on cell cycle activity in the neutrophil activated cancer cells.

FIG. 6C shows expression level of cell cycle related gene and protein was evaluated by PCR and Western blotting in the neutrophil and PLAG treated cells.

FIG. 6D shows verification of protein expression and phosphorylation related with PAR2 degradation in the PLAG and neutrophil co-treated activated cancer cells by western blot analysis.

FIG. 6E shows identification of PAR2 binding proteins using immunoprecipitation assay.

FIG. 6F shows degradation of PAR2 was verified with ubiquitin activity in the PLAG and neutrophil co-treated cancer cells by ubiquitination assay with anti-PAR2 antibody.

FIG. 7 shows an exemplary experimental design to investigate the synergistic effect of PLAG on PD-L1 immune-checkpoint drug therapy in the MB49 bladder cancer model in Example 3.

FIG. 8A shows analysis of tumor size change in each group estimate 3 days interval.

FIG. 8B shows changes in morphology and tumor size of mice on the day of sacrifice.

FIG. 8C shows tumor weight analysis in PLAG or aPD-L1 co-treat mice evaluated at the sacrificed day.

In FIGS. 8A-8C: Compared to the positive control: #P<0.05, ###<0.001; Compared with the aPD-L1 only treat group: $P<0.05, $$$P<0.001 (each experiment n=6). N.S, Not significant. Mean±SD

FIG. 9A shows PLAG modulating neutrophil count via complete blood count (CBC) analysis.

FIG. 9B shows analysis of blood Ly6G and CD11b positive cell sorting results according to PLAG and aPD-L1 treatment.

FIG. 9C shows analysis of tissue infiltrated Ly6G and CD11b positive cell sorting results according to PLAG and aPD-L1 treatment.

FIG. 9D shows the graphs represented FIG. 9B (Blood) and FIG. 9C (Tumor).

FIG. 9E show analysis of neutrophil infiltration effect by PLAG treatment in tumor tissue through IHC staining. (Ly6G: neutrophil population; Neutrophil Elastase: active neutrophil).

In FIGS. 9A-9E: Compared with the negative control: ***P<0.001; Compared with the positive control: #P<0.05, ##P<0.01, ###P<0.001; Compared with the aPD-L1 only treat group: $$P<0.01, $$$P<0.001 (each experiment n=3). N.S, Not significant. Mean±SD.

FIG. 10A shows PLAG modulating lymphocyte count via complete blood count (CBC) analysis.

FIG. 10B shows quantitative analysis of NLR levels in blood according to PLAG treatment.

FIGS. 10C-10D show analysis of blood CD4 and CD8 positive cell sorting results according to PLAG and aPD-L1 treatment.

FIGS. 10E and 10F show analysis of tumor tissue infiltrated CD4 and CD8 positive cell sorting results according to PLAG and aPD-L1 treatment.

FIG. 10G shows analysis of lymphocyte infiltration effect by PLAG treatment in tumor tissue through IHC staining.

In FIGS. 10A-10F: Compared with the negative control: ***P<0.001; Compared with the positive control: #P<0.05, ##P<0.01, ###P<0.001; Compared with the aPD-L1 only treat group: $$P<0.01, $$$P<0.001 (each experiment n=3). N.S, Not significant. Mean±SD.

FIGS. 11A-11B show that chemokine and growth factor secretion changes involved in neutrophil infiltration and activity.

FIGS. 11C-11D show that chemokine and cytokine secretion changes involved in T-cell polarity.

FIG. 11E shows that cytokine secretion changes involved in lymphocyte formation and activity.

In FIGS. 11A-11E: Compared with the negative control: *P<0.05 P<0.01***P<0.001; Compared with the positive control: #P<0.05, ##P<0.01, ###P<0.001; Compared with the aPD-L1 only treat group: $P<0.05, $$P<0.01, $$$P<0.001 (each experiment n=6). N.S, Not significant. Mean±SD.

FIG. 12 shows an exemplary experimental design to investigate the synergistic effect of PLAG on PD-L1 immune-checkpoint inhibitor treatment in the LLC-1 model in Example 4.

FIG. 13A shows analysis of tumor size change in each group estimate 3 days interval.

FIG. 13B shows changes in morphology and tumor size of mice on the day of sacrifice.

FIG. 13C shows tumor weight analysis in PLAG or aPD-1 co treat mice evaluated at the sacrificed day

In FIGS. 13A-13C: Compared to the positive control: ###P<0 001; Compared with the aPD-1 only treat group: $P<0.05, $$P<0.05, $$$P<0.001 (each experiment n=6). N S, Not significant. Mean±SD.

FIG. 14A shows PLAG modulating immune-cell count via complete blood count (CBC) analysis.

FIG. 14B shows analysis of blood/tumor CD4 or CD8 positive cell sorting results according to PLAG and aPD-1 treatment.

FIG. 14C shows analysis of blood/tissue infiltrated Ly6G positive cell sorting results according to PLAG and aPD-1 treatment.

FIG. 14D shows analysis of neutrophil infiltration control effect by PLAG treatment in tumor tissue through IHC staining.

In FIG. 14A: Compared with the negative control: ***P<0.001; Compared with the positive control: #P<0.05, ##P<0.01, ###P<0.001; Compared with the aPD-1 only treat group: $$P<0.01, $$$P<0.001 (each experiment n=6). N.S, Not significant. Mean±SD.

FIGS. 15A-15B show analysis of blood/tumor Th17 cell population sorting results according to PLAG and aPD-1 treatment.

In FIG. 15B, Compared with the negative control: ***P<0.001;Compared with the positive control: ###P<0.001; Compared with the aPD-1 only treat group: $P<0.01 (each experiment n=3). N.S, Not significant. Mean±SD.

FIG. 16A shows changes in morphology and tumor size of mice on the weekly sacrifice.

FIG. 16B shows compounds modulating immune-cell count via complete blood count (CBC) analysis.

FIG. 16C shows analysis of blood/tissue infiltrated Ly6G positive cell sorting results according to compounds treatment.

FIG. 16D shows analysis of blood/tumor Th 17 cell population sorting results according to PLAG and aPD-1 treatment.

In FIGS. 16A-16D: each experiment n=6. Navarixin: CXCR 2 antagonist; aLy6G: anti-Ly6G antibody treatment. Compared with the negative control: ***P<0.001; Compared with the positive control: #P<0.05, ##P<0.01, ###P<0.001; Compared with the aPD-1 only treat group: $$P<0.01, $$$P<0.001 (each experiment n=6). N.S, Not significant. Mean±SD.

FIG. 17A shows analysis of adenosine concentration in plasma on sacrifice day according to PLAG and aPD-1 treatment.

FIG. 17B shows analysis of adenosine concentration in plasma on weekly sacrificed according to compounds treatment. Navarixin: CXCR2 antagonist; aLy6G: anti-Ly6G antibody treatment. Compared with the negative control: ***P<0.001; Compared with the positive control: #P<0.05, ##P<0.01, ###P<0.001; Compared with the aPD-1 only treat group: $$P<0.01, $$$P<0.001 (each experiment n=6). N.S, Not significant. Mean±SD.

### DETAILED DESCRIPTION

The terms PLAG, EC-18 and 1-palmitoyl-2-linoleoyl-3-acetylglycerol are used interchangeably herein and designate the same compound herein.

### Definitions

The abbreviations used herein have their conventional meaning within the chemical and biological arts. The chemical structures and formulae set forth herein are constructed according to the standard rules of chemical valency known in the chemical arts.

It will be apparent to one skilled in the art that certain compounds disclosed herein may exist in tautomeric forms, all such tautomeric forms of the compounds being within the scope of the invention.

The terms “a” or “an,” as used in herein means one or more. For example, the singular forms “a,” “an” and “the” are intended to include the plural forms as well, unless the context clearly indicates otherwise. It will be further understood that the terms “comprise”, “include”, “have”, etc. when used in this specification, specify the presence of stated features, regions, integers, steps, processes, operations, elements and/or components but do not preclude the presence or addition of one or more other features, regions, integers, steps, processes, operations, elements, components, and/or combinations thereof.

Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. The term “treating” and conjugations thereof, may include prevention of an injury, pathology, condition, or disease. In embodiments, treating is preventing. In embodiments, treating does not include preventing.

The term “prevent” refers to a decrease in the occurrence of disease symptoms in a patient. As indicated above, the prevention may be complete (e.g., no detectable symptoms) or partial, such that fewer symptoms are observed than would likely occur absent treatment.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals In some embodiments, a patient or subject is human.

An “effective amount” or a “therapeutically effective amount” is an amount sufficient for a compound to accomplish a stated purpose relative to the absence of the compound (e.g. achieve the effect for which it is administered, treat a disease, reduce enzyme activity, increase enzyme activity, reduce a catabolic enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the tem “in combination” does not restrict the order in which the therapies (e.g., a first and second therapy) are administered to a subject. A first therapy (e.g. administration of either i) one or more chemotherapeutic agents or ii) a monoacetyl diacylglycerol compound of Formula (I) such as PLAG) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours or up to about one 1 week before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours or up to about one 1 week after) the administration of a second therapy (e.g. administration of either i) a monoacetyl diacylglycerol compound of Formula (I) such as PLAG or ii)) one or more chemotherapeutic agents) to a subject which had, has, or is susceptible to cancer, including a subject that has been diagnosed with a solid tumor. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The terms “proliferative disorder” and “proliferative disease” refer to disorders associated with abnormal cell proliferation such as cancer.

“Tumor” and “neoplasm” or similar term as used herein refer to any mass of tissue that result from excessive cell growth or proliferation, either benign or malignant including pre-cancerous lesions.

By “neoplasia” is meant a disease or disorder characterized by excess proliferation or reduced apoptosis. Illustrative neoplasms for which the invention can be used include, but are not limited to cancers including solid tumors. Further illustrative neoplasms for which the invention can be used include, but are not limited to bladder cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, gastric and esophageal cancer, head and neck cancer, rectal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In particular embodiments, the neoplasia is multiple myeloma, beta-cell lymphoma, urothelial/bladder carcinoma or melanoma.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and me thylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL™ paclitaxel, ABRAXANE™ Cremophor-free, albumin-engineered nanoparticle formulation of paclitaxel, and TAXOTERE™ doxetaxel; chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts or acids of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™); and (xi) pharmaceutically acceptable salts or acids of any of the above.

As dicussed, in certain aspects, preferred chemotherapeutic agents include immune checkpoint inhibitor agents. For instance, suitable and preferred checkpoint inhibitors for use in the present methods and compositions include inhibitors of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H11, B7H3, B7H4, BTLA, CD2, CD27, CD28, CD30, CD40, CD70, CD80, CD86, CD137, CD160, CD226, CD276, DR3, GAL9, GITR, HAVCR2, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of IDO1, CTLA4, PD-1, LAG3, PD-L1, TIM3, or combinations thereof.

In certain preferred compositions and methods, the immune checkpoint inhibitor is an inhibitor of PD-L1. In certain preferred compositions and methods some embodiments, the immune checkpoint inhibitor is an inhibitor of PD-1, in certain preferred compositions and methods, the immune checkpoint inhibitor is an inhibitor of CTLA-4. In certain preferred compositions and methods, the immune checkpoint inhibitor is an inhibitor of LAG3. In certain preferred compositions and methods, the immune checkpoint inhibitor is an inhibitor of TIM3. In certain preferred compositions and methods, the immune checkpoint inhibitor is an inhibitor of IDO1.

The present methods and compositions can effectively reduce or suppress tumor growth in a patient, for example a cancer patient that receives a therapy of a) one or more chemotherapeutic agents and b) a monoacetyl diacylglycerol compound of Formula (I) such as PLAG. Co-treatment with PLAG and one or more distinct chemotherapeutic agents may result in a 5, 10, 15, 20, 25, 30, 35, 40, 45 or 50 percent or more reduction in tumor volume.

A wide variety of type of cancers may be treated in accordance with the present methods and compositions. For instance, a cancer to be treated may be a solid tumor. Illustrative cancers for which the invention can be used include, but are not limited to breast cancer, leukemias (e.g., kemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, ovarian cancer, prostate cancer, gastric and esophageal cancer, head and neck cancer, rectal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

A chemical synthetic method for the preparation of monoacetyldiacylglycerol compounds of Formula (I) is shown, for example, in Korean Registered Patents No. 10-0789323 and No. 10-1278874, the contents of which are incorporated herein by reference. For example, PLAG can be synthesized by acylating the hydroxy groups of glycerol with acetyl, palmitoyl and linoleoyl functional groups.

Therapeutically effective amounts of a monoacetyl diacylglycerol compound of Formula (I) such as PLAG and one or more distinct chemotherapeutic agents can be initially determined from cell culture assays. Target concentrations will be those concentrations of active compound(s) that are capable of achieving the methods described herein, as measured using the methods described herein or known in the art. Treatment amounts of a monoacetyl diacylglycerol compound of Formula (I) such as PLAG and one or more distinct chemotherapeutic agents also have been previously reported.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The frequency of administration of the composition of the present invention is not particularly limited, but it may be administered once a day or several times a day with divided dosage.

An exemplary daily dosages for a patient such as a human in need of treatment of a compound of Formula (I) or Formula (II) (i.e. EC-18) include between 0.0001 mg/kg and 4 mg/kg body weight, or between 0.01 mg/kg and 4 mg/kg body weight e.g., up to or about 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight of the subject such as a human patient in need thereof. For example, in some embodiments, an effective weekly dose of a compound of Formula (I) or Formula (II) may between 0.1 μg/kg body weight and 400 μg/kg body weight such as a human patient in need thereof. In preferred aspects, an oral formulation is utilized such as a tablet or capsule (e.g. soft gelatin capsule) that contains 250-1000 mg, e.g., 500 mg, of a compound of Formula (I) or Formula (II). Optimal dosage amounts also can be determined empirically for particular patients or identified group of patients.

Exemplary effective daily doses of the distinct chemotherapeutic agent(s) also can vary and can be determined empirically for particular patients or identified group of patients. If the agent has been clinically used for anti-cancer therapies, in one aspect, the agent cab e use din same or similar dosage amounts as the agent has been used previously without EC-18. In certain aspects, exemplary daily doses of the distinct chemotherapeutic agent(s) may be between, for example, 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

A monoacetyl diacylglycerol compound of Formula (I) such as PLAG and one or more chemotherapeutic agents can be administered to a subject by any of a number of routes such as topical contact, oral, intravenous, intraperitoneal, intramuscular, intralesional, intrathecal, intranasal or subcutaneous administration, or the implantation of a slow-release device, e.g., a mini-osmotic pump, to a subject. Parenteral administration includes, e.g., intravenous, intramuscular, intra-arteriole, intradermal, subcutaneous, intraperitoneal, intraventricular, and intracranial.

Pharmaceutical compositions may include compositions wherein one or both of a monoacetyl diacylglycerol compound of Formula (I) such as PLAG and one or more chemotherapeutic agents is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

Pharmaceutical composition may be manufactured with additional pharmaceutically acceptable carrier for each formulation. As used herein, the term “pharmaceutically acceptable carrier” may refer to a carrier or diluent that does not stimulate organism and not inhibiting biological activity and characteristic of the injected compound. The type of the carrier that can be used in the present invention is not particularly limited, any carrier conventionally used in the area of industry and pharmaceutically acceptable may be used.

Saline, sterilized water, IV fluids, buffer saline, albumin injection solution, dextrose solution, maltodextrin solution, glycerol, ethanol are non-limiting examples of the usable carriers. These carriers may be used alone or in combination of two or more. The carrier may include a non-naturally occurring carrier. If necessary, other conventionally used additives like an antioxidant, a buffer and/or a bacteriostatic agent may be added and used. It may be formulated with diluent, a dispersant, a surfactant, a bonding agent, a lubricant to make an injection solution like aqueous solution, suspension, emulsion, and pills, capsules, granules or tablets, and the like.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

As discussed, kits are also provided. For instance, in this aspect, a monoacetyl diacylglycerol compound of Formula (I) such as PLAG and one or more chemotherapeutic agents each suitably can be packaged in suitable containers labeled for a specified treatment. The containers can include a PLAG compound or composition, one or more chemotherapeutic agents and one or more of a suitable stabilizer, carrier molecule and/or the like, as appropriate for the intended use. In other embodiments, the kit further comprises one or more therapeutic reagents for an intended treatment, such as one or more additional chemotherapeutic agents. A product can include a container (e.g., a vial, jar, bottle, bag, or the like) containing a PLAG compound or composition and/or one or more chemotherapeutic agents. In addition, an article of manufacture or kit further may include, for example, packaging materials, instructions for use, syringes, delivery devices, for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

As discussed, a monoacetyl diacylglycerol compound such as PLAG and one or more distinct chemotherapeutic agents suitably are administered in a coordinated manner, for example either simultaneously or sequentially. For instance, a monoacetyl diacylglycerol compound such as PLAG and one or more distinct chemotherapeutic agents may be administered to a subject at substantially the same time, or the agents instead may be administered to the subject at different times, suitably within hours although longer periods between the separate administrations also may be suitable.

### EXAMPLES

Although the foregoing section has been described in some detail by way of illustration and example for purposes of clarity of understanding, it is apparent to those skilled in the art that certain minor changes and modifications will be practiced in light of the above teaching Therefore, the description and examples should not be construed as limiting the scope of any invention described herein.

All references cited herein, including patent applications and publications, are hereby incorporated by reference in their entirety.

### Example 1: Anti-Cancer Effects of PLAG in Combination with AC-Regimen in a Xenograft Mouse Model

MDA-MB-231 breast cancer Xenograft model was used. Tumor growth was evaluated in the AC-regimen alone and PLAG co-treated animals. AC-regimen was delivered via IP injection twice a week with dose of 2/20 and 5/50 mph (Doxorubicin/Cyclophosphamide), and PLAG was daily administered with 100 and 250 mpk. Tumor growth was calculated with 3 day intervals. Neutrophil chemotaxis related chemokines, CXCL1/2/8 and circulating neutrophils were also evaluated with 2 week internal. Expression of apoptosis molecular markers, Bax/Bak and tumor-infiltrating neutrophil (TIN) in the tumor lesion was analyzed by immunohistochemistry (IHC).

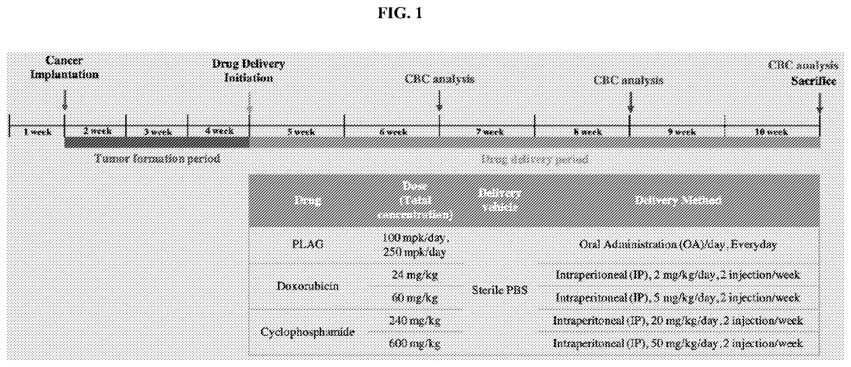

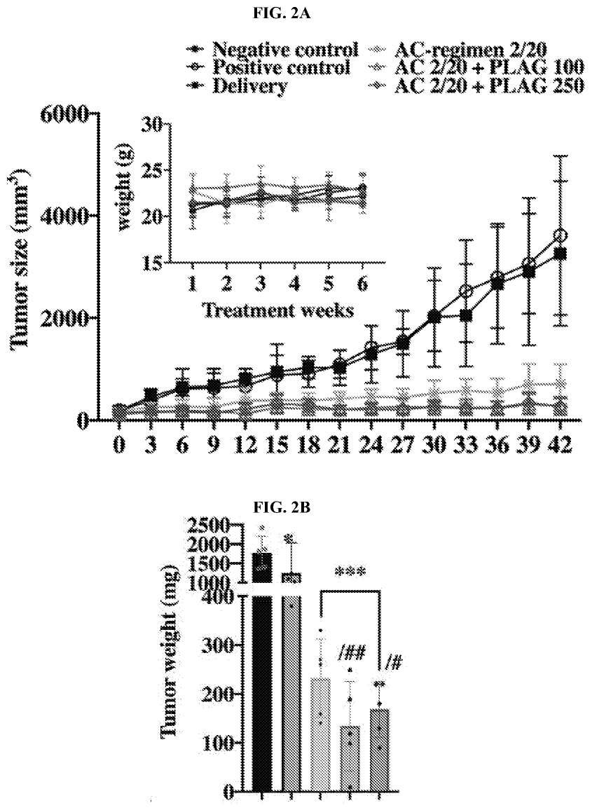

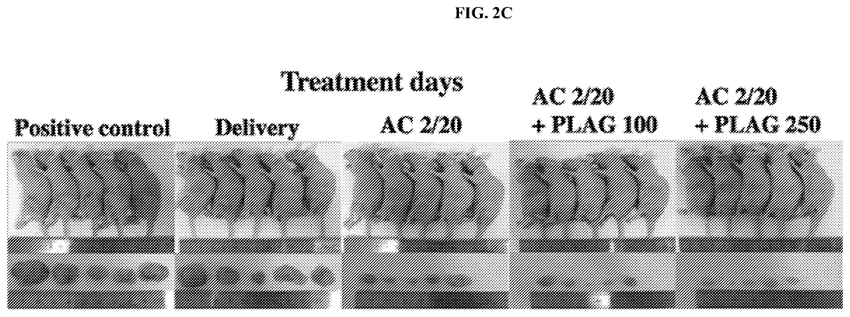

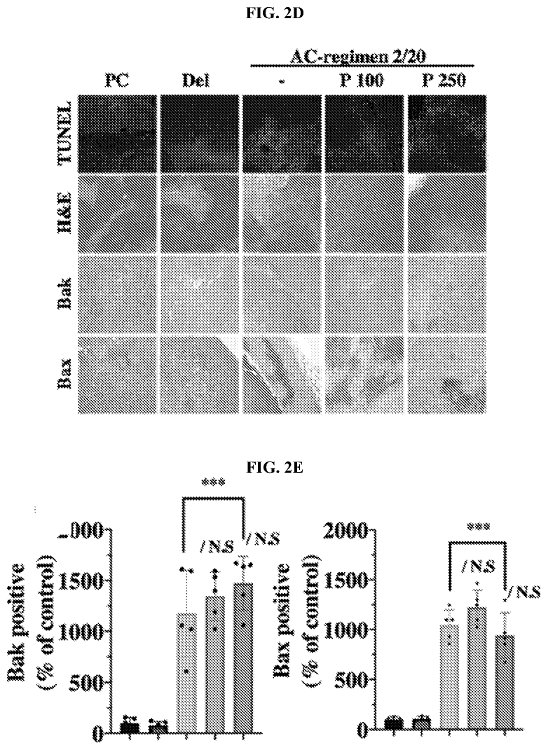

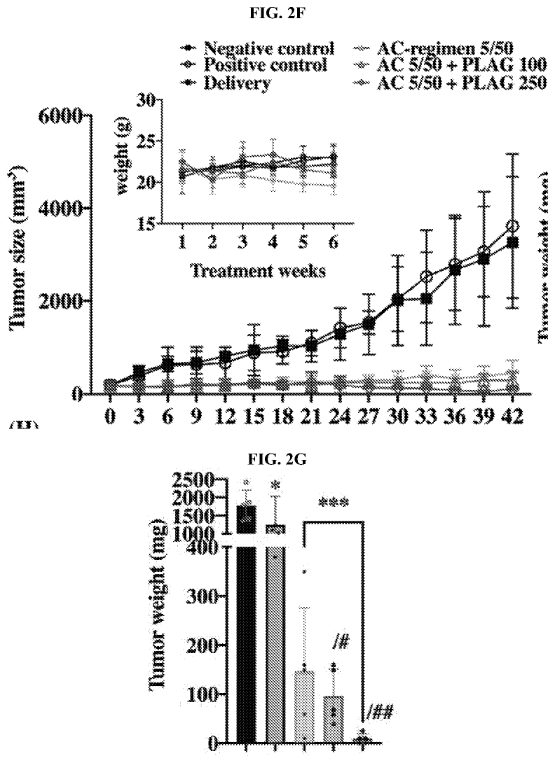

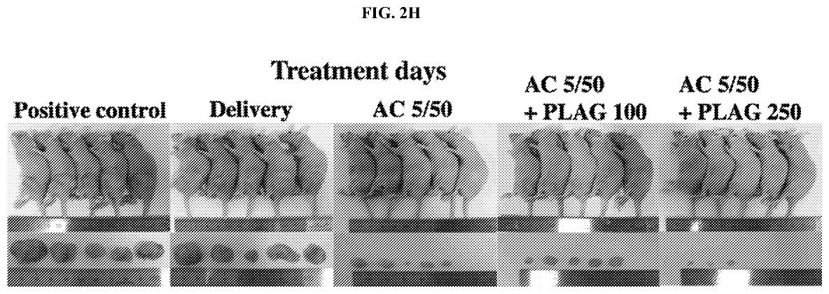

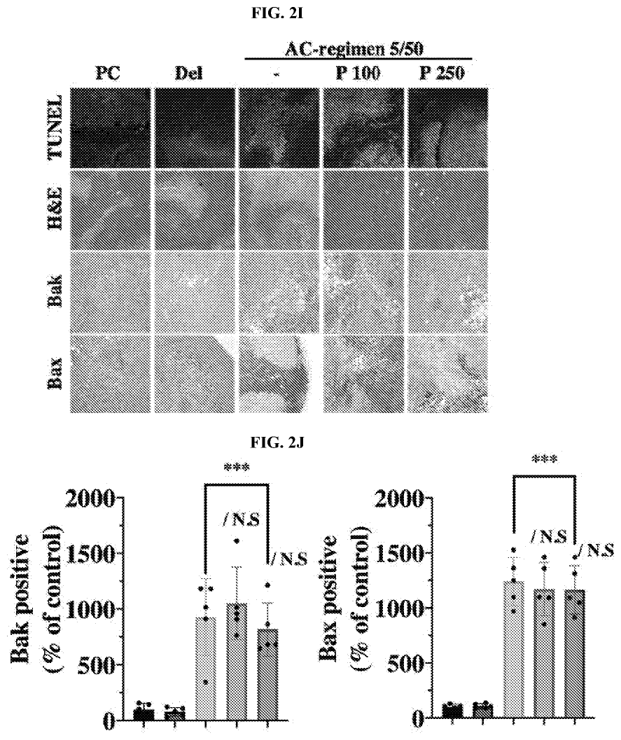

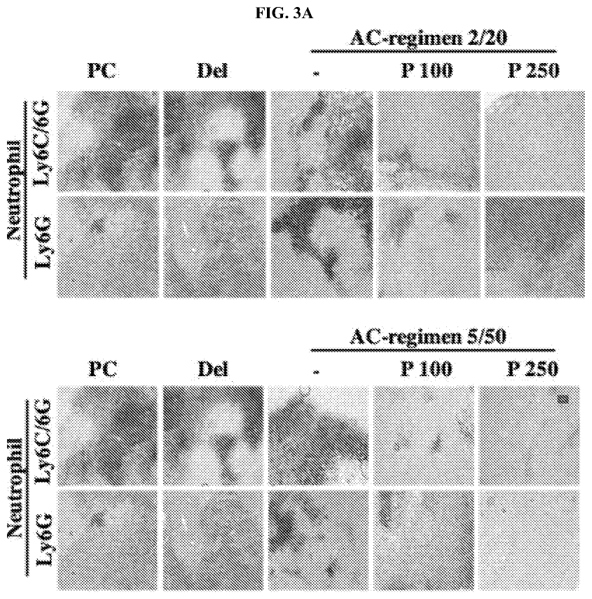

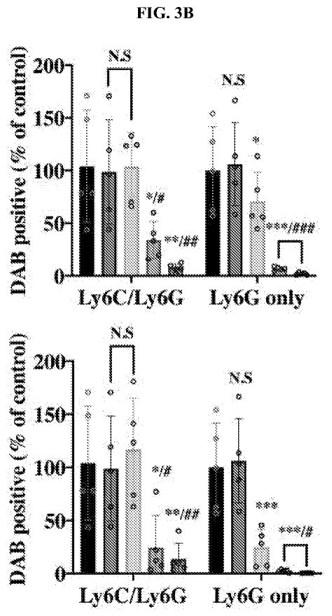

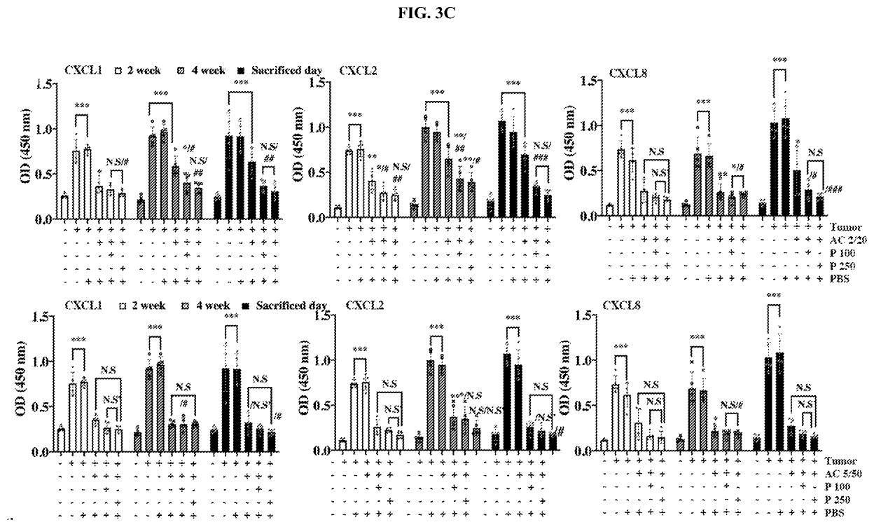

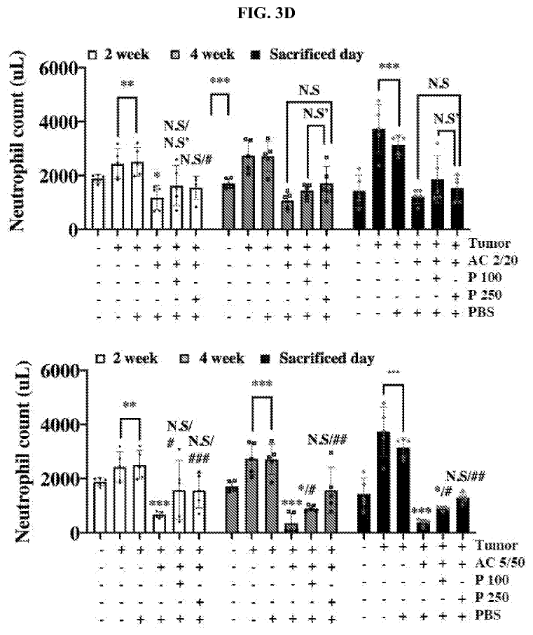

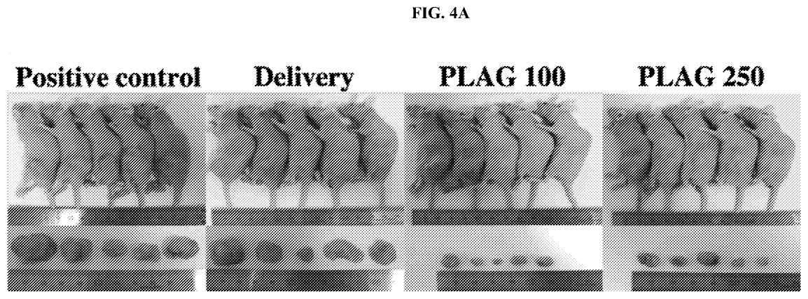

PLAG has synergistic effects decreasing the tumor burden in the PLAG and AC-regimen co-regimen co-treated Xenograft mice. In AC-regimen with 2/20 or 5/50 mpk treated groups, retardation of tumor growth was observed by calculating tumor size and processed apoptosis was proved by TUNEL and apoptosis-related proteins expression in the regressed tumor burden with AC-regimen. Modulated chemokine expression from tumor burden and subsequent neutrophil recruitment were also detected with dependent on tumor mass. In the PLAG co-treated group, smaller tumor burden than that in AC-regimen alone was consistently observed until sacrifice. It was also confirmed that the tumor burden of the PLAG co-treated group with 5/50 AC-regimen was significantly decreased in a concentration-dependent manner compared to the AC-regimen along group (p<0.05). Especially, in 250 mpk PLAG co-treated group, no tumor tissues were found in tests mice on the sacrifice day. Significantly reduced chemokine expression and TIN in the PLAG added group were proved through IHC and chemokine analysis. Additionally, interruption of tumor growth, and reduced chemokine expression and TIN were observed in the PLAG alone treated groups. In summary, PLAG has a synergistic effect on regression of tumor burden in the subjects co-treated with AC-regimen. Analysis of tumor tissue with IHC revealed that TIN was dramatically reduced in the PLAG cotreated group. Results also are set forth in FIGS. 1, 2 (includes FIGS. 2A-2J) and 3(includes FIGS. 3A-3D).

### Example 2: Anti-Cancer Effects of PLAG

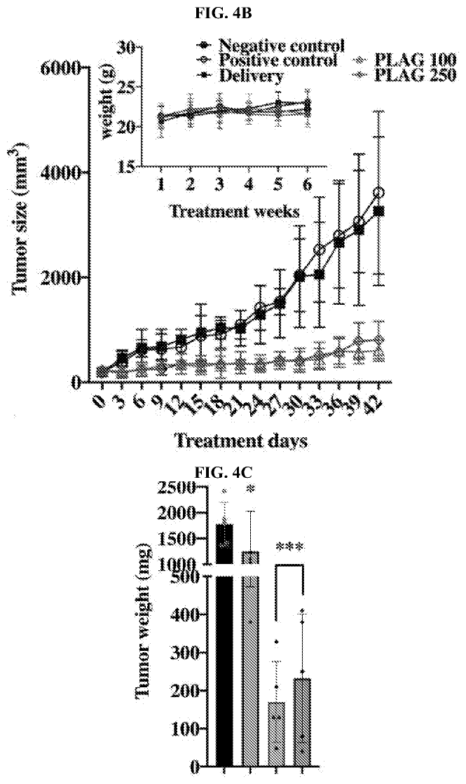

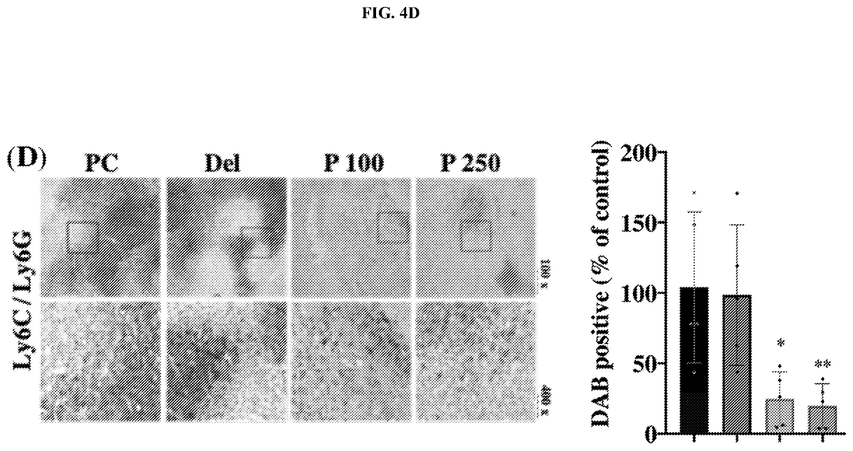

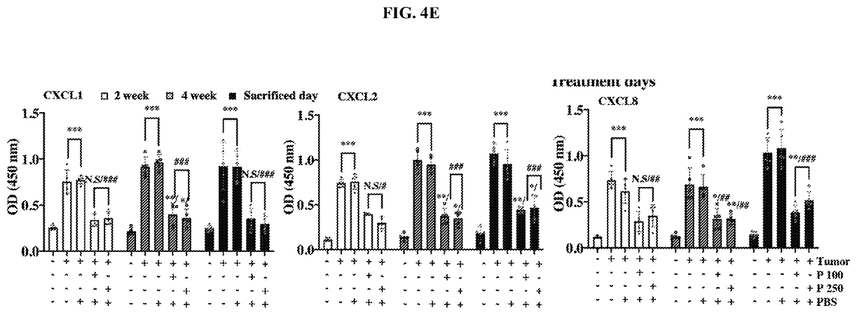

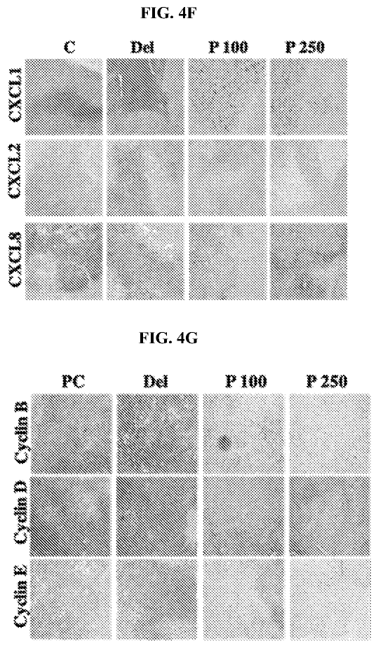

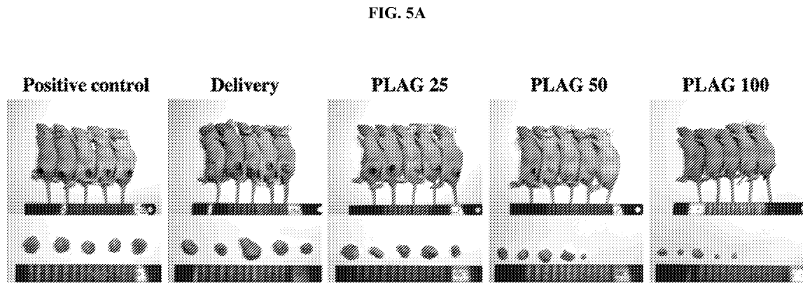

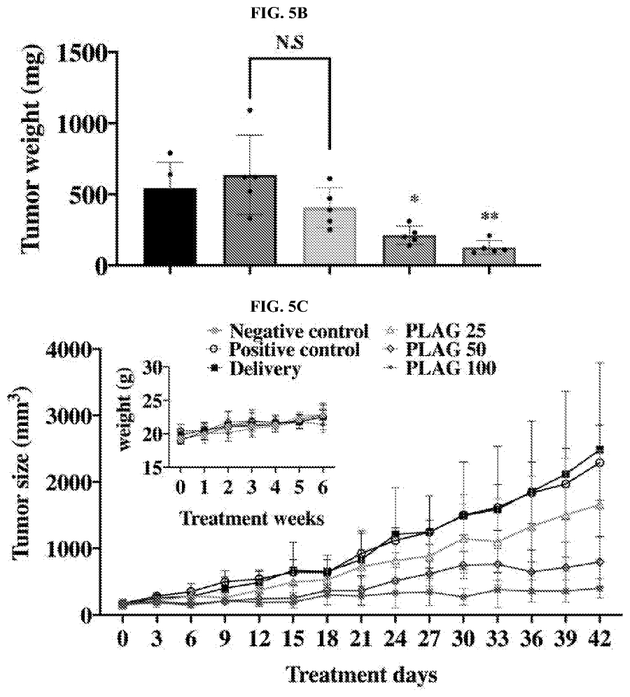

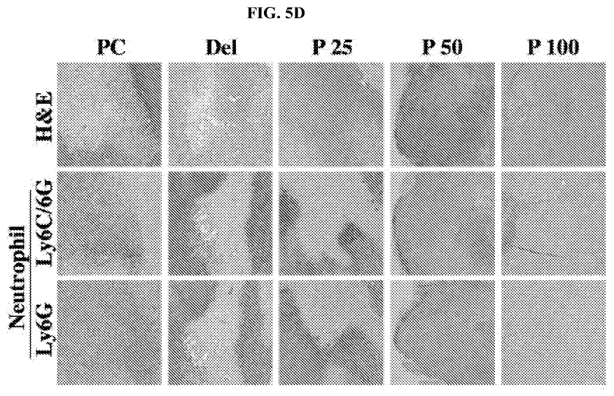

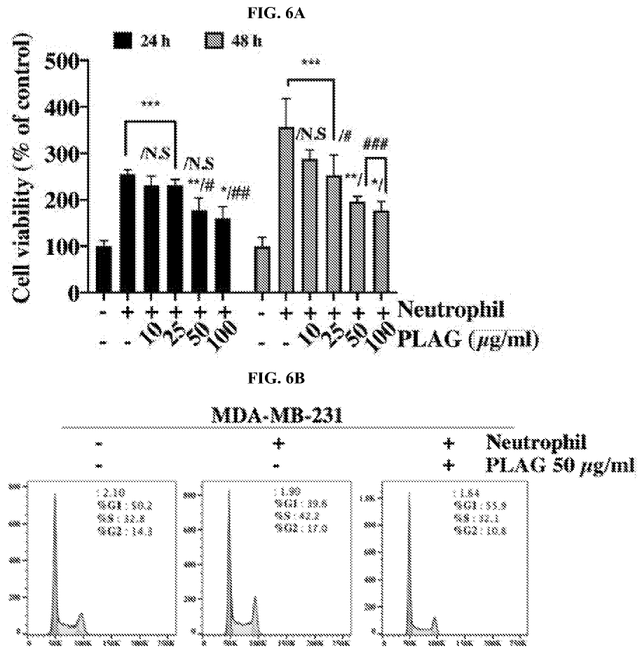

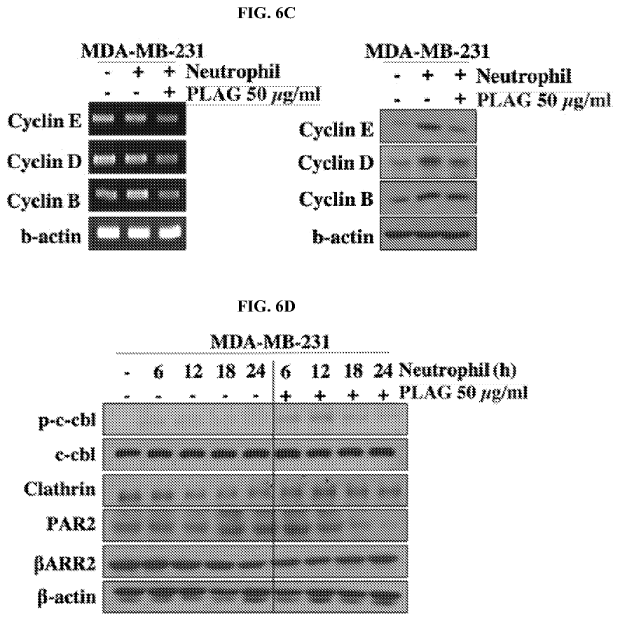

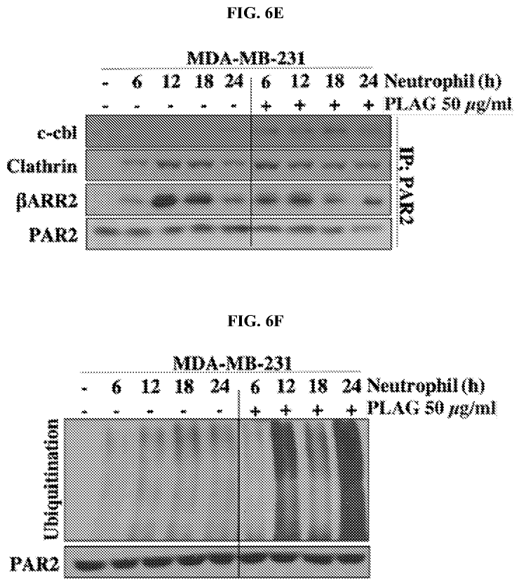

FIGS. 4 (includes FIGS. 4A-4G), 5 (includes FIGS. 5A-5D) and 6 (includes FIGS. 6A-6B) show treatment of cancer with PLAG. These results show treatment with PLAG can effectively regulate the expression of neutrophil chemotactic chemokine in cancer tissues to control excessive infiltration of neutrophils. These results show that PLAG administered alone (without additional chemotherapeutic agents) can regulate excessive growth by inhibiting the cell cycle of cancer tissues. These results also show that administration of PLAG can inhibit cell cycle and chemokine expression by inducing PAR2 degradation in cancer tissues.

### Example 3: The Effect of PLAG on the PD-L1 Immune-Checkpoint Drug Therapy in the MB49 Bladder Cancer Syngeneic Model

The development of immune-checkpoint drugs, a fourth-generation anticancer drug, is a significant breakthrough in anticancer research. Cancer therapy using immune-checkpoint drugs are still enlarged. To improve the efficacy of immunecheckpoint drugs, modulation of the tumor microenvironment is essentially required. In this experiment, we examined the elevated anti-cancer efficacy of 1-Palmitoyl-2-Linoleoyl-3-Acetyl-rac-Glycerol (PLAG), which has been demonstrated to attenuate tumor infiltrating neutrophils (TINS) in the tumor, along with the PD-L1 immune checkpoint inhibitor treatment.

### Methods

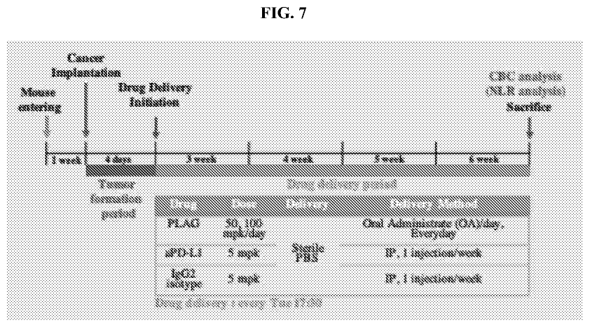

The syngeneic model was used (n=6) to investigate the enhanced anti-tumor effect of PD-L1 antibody with the addition of PLAG. MB49 murine bladder cancer cells were implanted into the C57BL/6 mice subcutaneously and bred for 5 weeks. After a week from tumor implantation, PLAG at different dosages (50/100 mpk) were daily administered orally for another 4 weeks with or without 5 mpk PD-L1 antibody (10F.9G2). PD-L1 antibody was delivered via IP injection once a week. (FIG. 7, Table 1)

TABLE 1

| Compound concentration | Compound delivery |
|---|---|
| PLAG: 50, 100 mpk | O.A: PLAG (Daily) |
| PD-L1 immune-checkpoint | I.P: aPD-L1 (5 mpk, 1 injection/week) |
| inhibition | I.P: Isotype (5 mpk, 1 injection/week) |
| antibody (aPD-L1): 5 mpk | |
| (BioXcell, 10F.9G2 clone) | |
| IgG2 isotype antibody: 5 mpk | |
| (BioXcell) | |

### Results

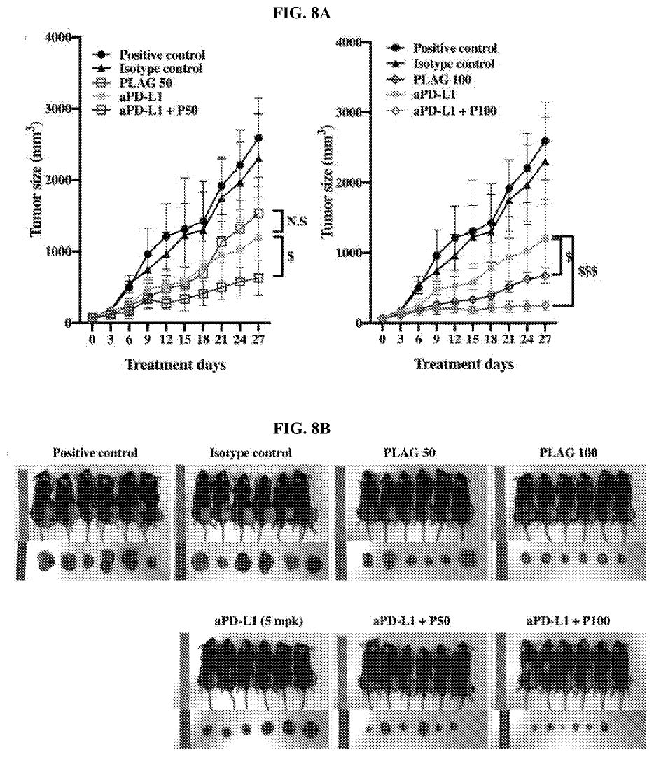

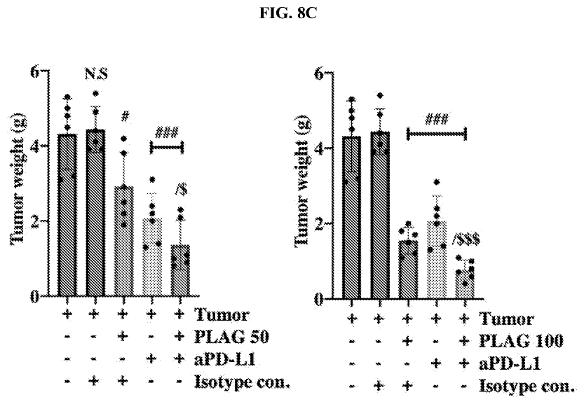

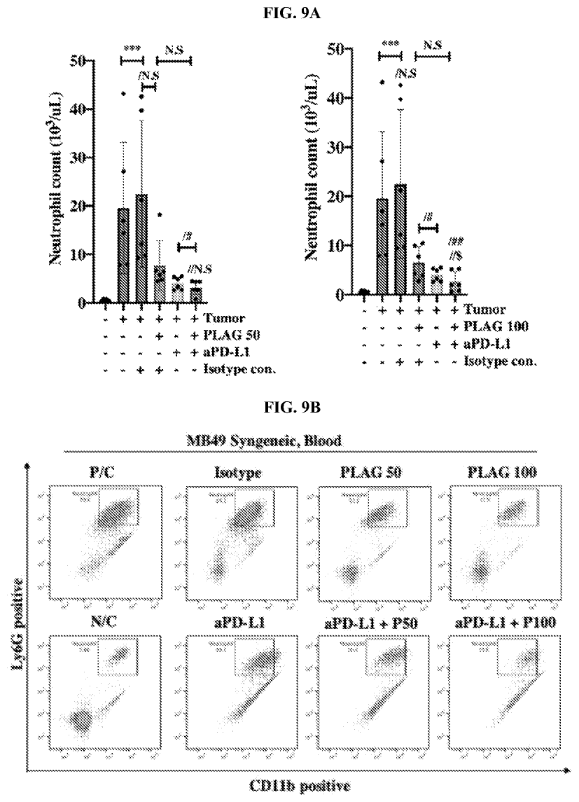

The PLAG treatment groups demonstrated that the tumor burden decreased in a concentration-dependent manner. In 50 and 100 mpk of PLAG treated mice, the tumor burden was decreased to a significant value compared to a positive control (p<0.05). In the group treated with the PD-L1 antibody alone, the growth rate of the tumor decreased until about 2 weeks. In the group treated simultaneously with PLAG and PD-L1 antibody, the growth of the tumor was significantly reduced compared to the group treated with PD-L1 antibody alone. Increased inhibitory effect of aPD-L1 on tumor progression by PLAG treatment are shown in FIG. 8A-8C.

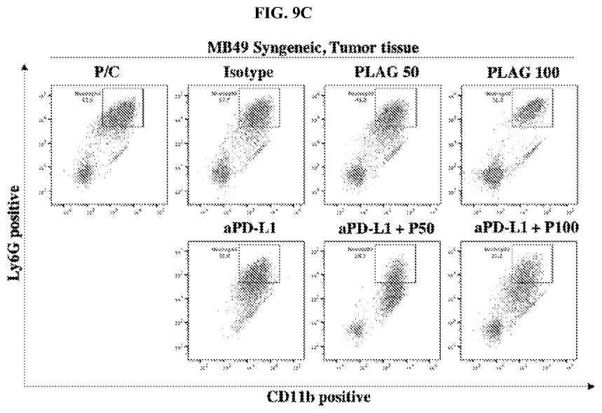

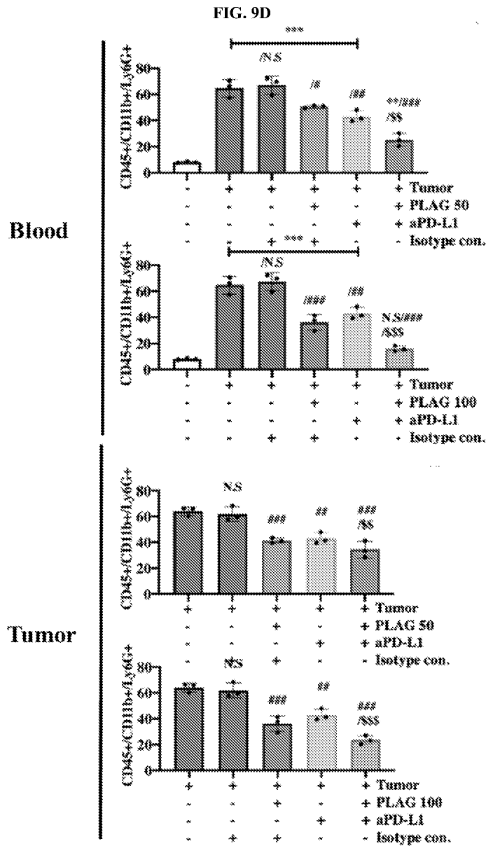

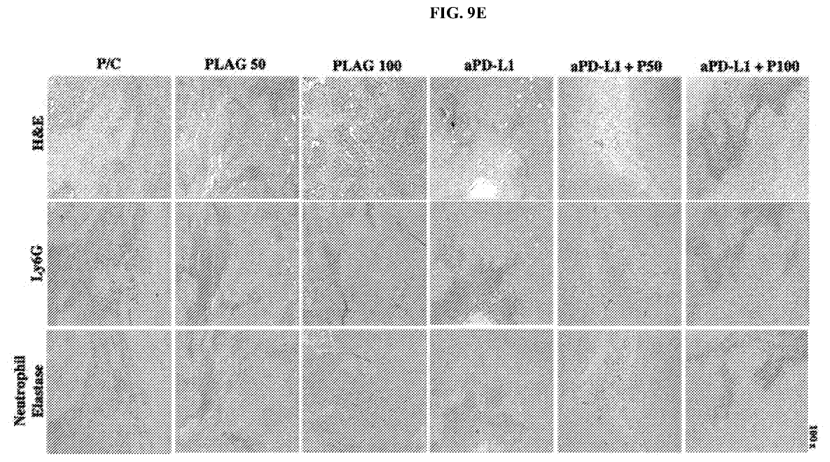

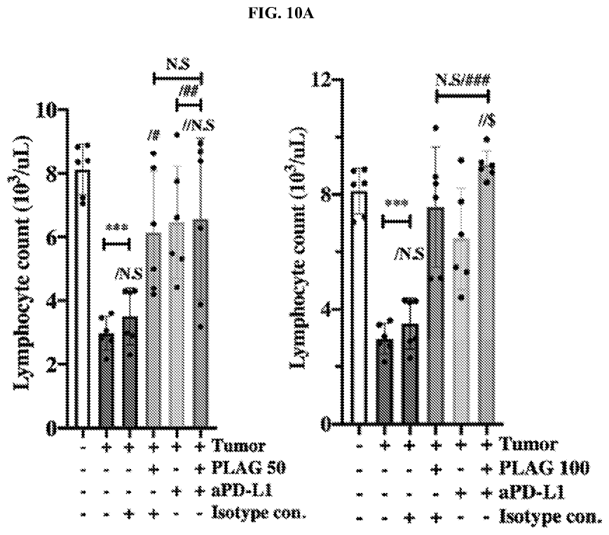

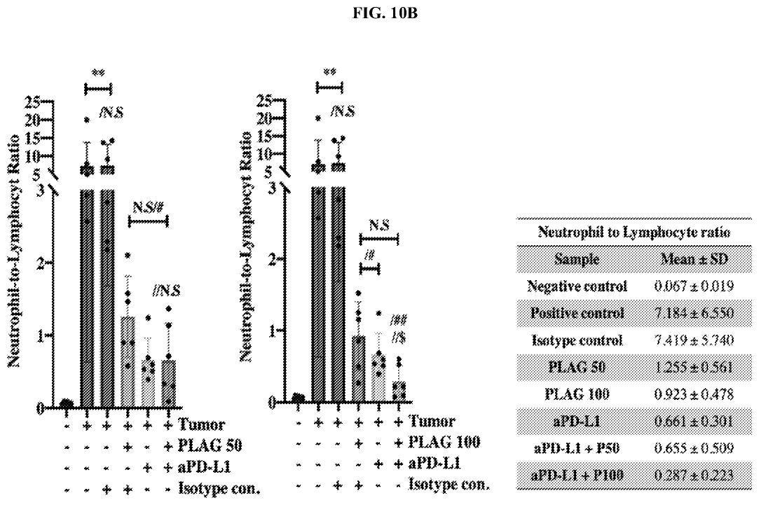

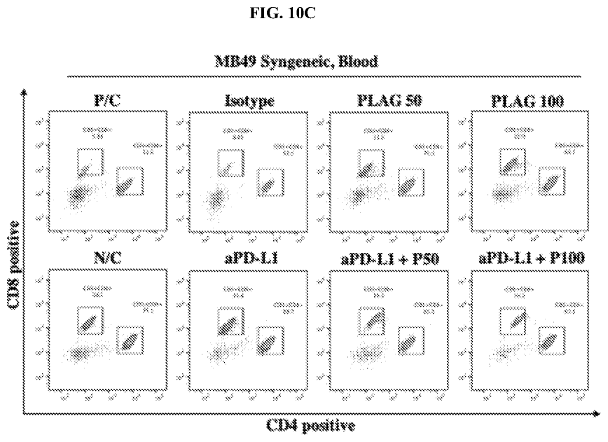

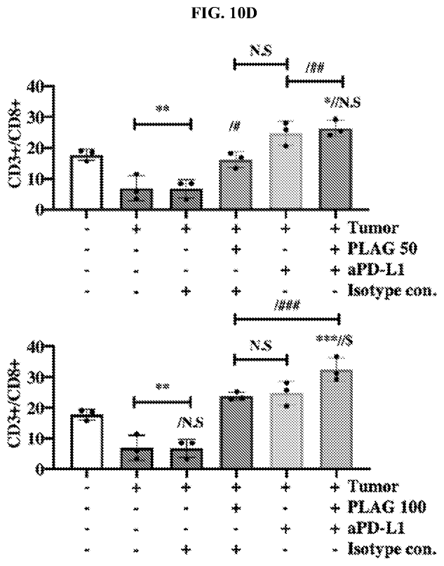

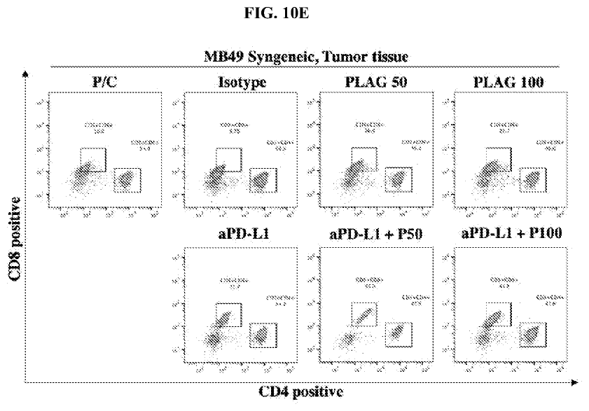

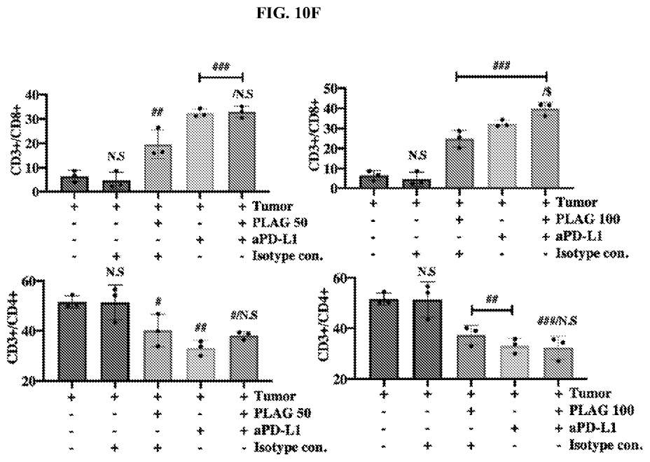

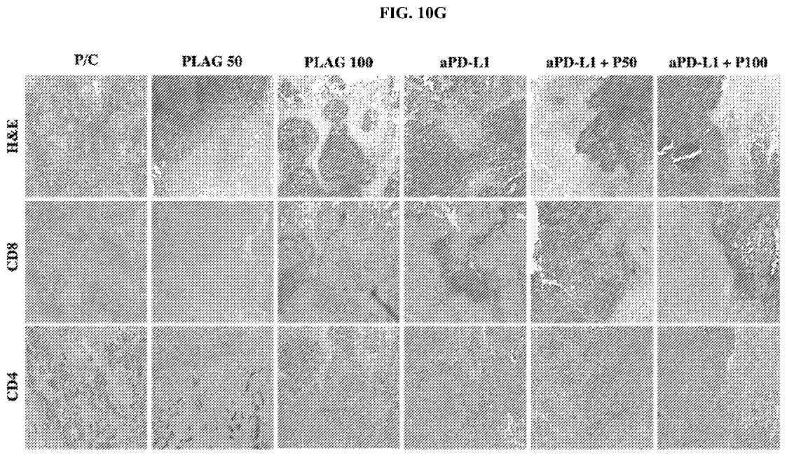

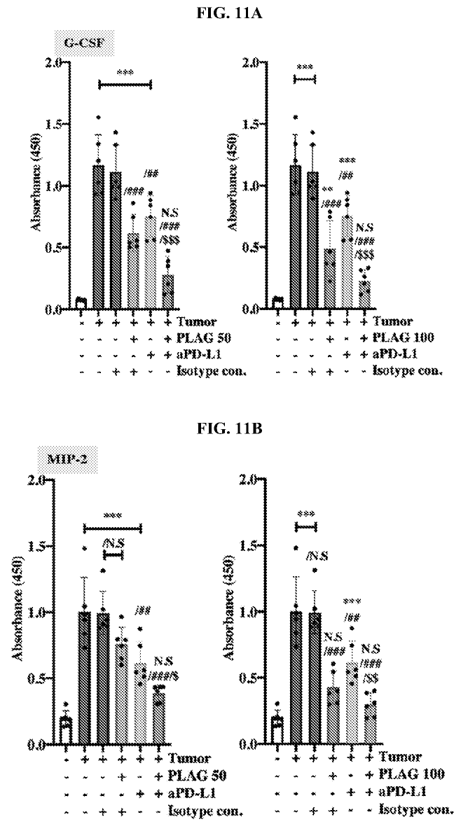

Control of neutrophil population and tumor infiltration by PLAG treatment is demonstrated in FIGS. 9A-9E and control of lymphocyte population and tumor infiltration by PLAG treatment is demonstrated in FIGS. 10A-10G. As a result of calculating neutrophils and lymphocytes every two weeks, the neutrophil-to-lymphocyte ratio (NLR) level in the group treated with PLAG and PD-L1 was significantly decreased compared to that treated with PD-L1 antibody alone. Besides, the number of TINs were effectively reduced by PLAG treatment alone.

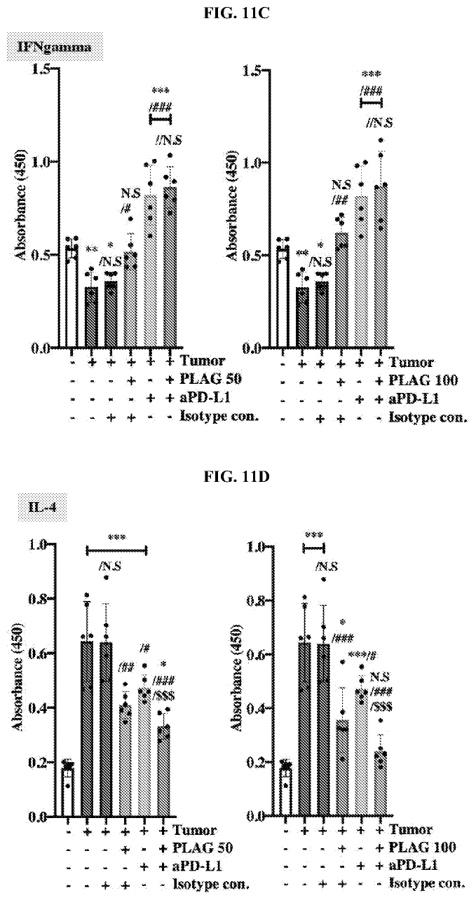

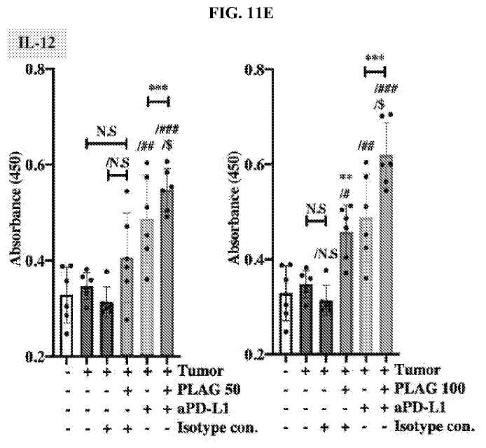

Validation of cytokine and chemokine secretion involved in immune cell population by PLAG treatment are demonstrated in FIGS. 11A-11E. The interruption of tumor growth and reduced chemokine secretion were also observed in the PLAG treated groups. In summary, our data suggest that PLAG provides an enhanced PD-L1 antibody effect on the regression of tumor burden in the syngeneic mice model via reducing the number of TINs.

### Conclusion

PLAG may be utilized for improving the efficacy of the PD-L1 antibody on reducing the tumor burden at the devastating tumor microenvironment. PLAG not only increases the anti-tumor effect of aPD-L1 more effectively, but it can suppress tumor progression on its own. In particular, tumor infiltrating neutrophils (TINs), which increases tumor progression, is effectively reduced by PLAG. Through effectively reducing the number of TINs by PLAG, the anti-tumoral effect by cytotoxic T-lymphocytes (CTLs) is further increased.

### Example 4: The Synergistic Anticancer Effect of PLAG on the PD-1 Immune-Checkpoint Inhibitor Treatment in the LLC-1 Syngeneic Model

### Background

Although immune checkpoint inhibitor (ICI) therapy usage has been increasing for various indications, some patients of various types of cancer were shown to not respond to ICI. To improve ICI response rate, a combination therapy targeting additional mechanisms to prevent tumor immune evasion by modulating the tumor microenvironment may be needed.

### Methods

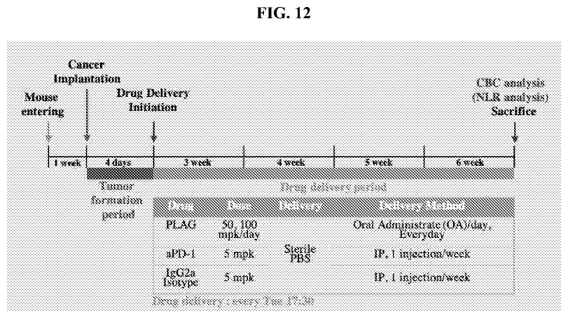

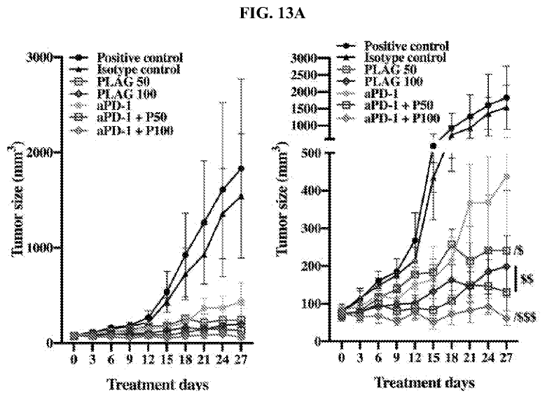

To investigate the enhanced anti tumor effect of the anti-PD-1 antibody (aPD-1 with the addition of 1-palmitoyl-2-linoleoyk-3-acetyl-rac-glycerol (PLAG), the syngeneic model was used (n=6/group), LLC-1 lung carcinoma was implanted into C57BL/6 mice subcutaneously. PLAG was daily administrated for 4 weeks with or without aPD-1 (RMP 1-14. aPD-1 was delivered via IP injection once a week. The degree of infiltrated lymphocyte population and neutrophils in the tumor and blood on the sacrificed day were analyzed (FIG. 12, Table 2).

TABLE 2

| Compound concentration | Compound delivery |
|---|---|
| PLAG: 50, 100 mpk | O.A: PLAG (Daily) |
| PD-L1 immune-checkpoint | I.P: aPD-L1 (5 mpk, 1 injection/week) |
| inhibition | I.P: Isotype (5 mpk, 1 injection/week) |
| antibody (aPD-L1): 5 mpk | |
| (BioXcell, RMP1-14 clone) | |
| IgG2 isotype antibody: 5 mpk | |
| (BioXcell) | |

### Results

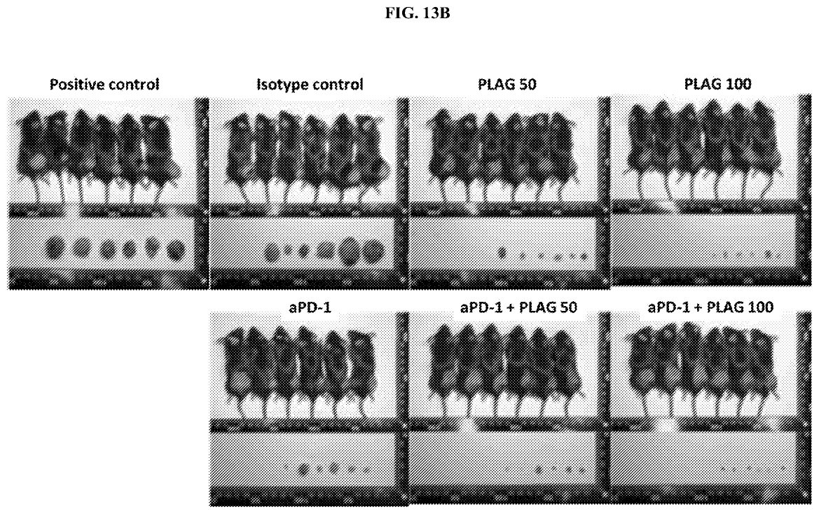

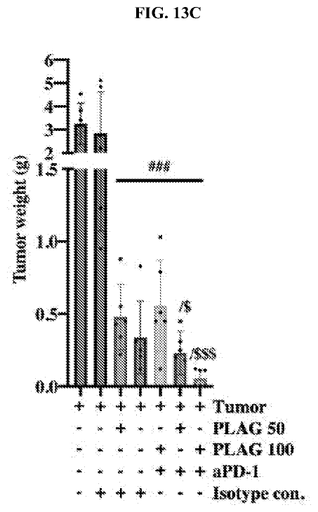

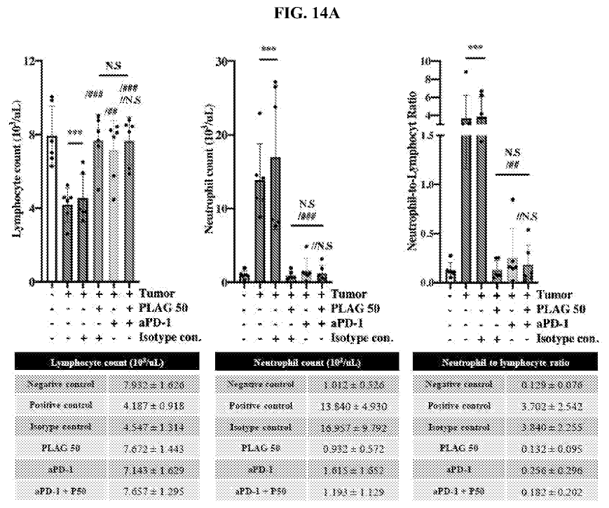

In PLAG treated 50 and 100 mpk mice group, the tumor burden was significantly reduced compared to a positive control p 0 05 In the group treated with aPD-1 alone, the tumor growth decreased by about 65 compared to the positive control. However, in mice co-treated with PLAG, the tumor was significantly reduced 18 compared to the aPD-1 alone. Synergistic anti tumor effect of PLAG with anti PD 1 antibody(aPD-1) is demonstrated in FIGS. 13A-13D.

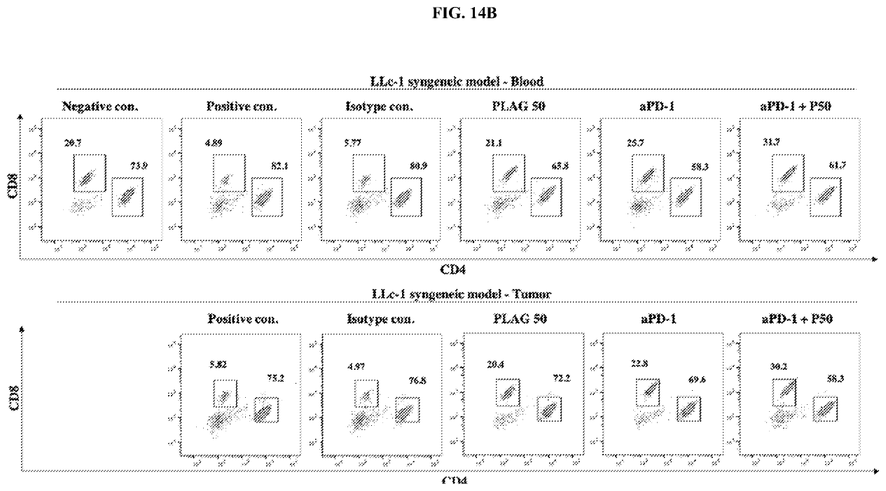

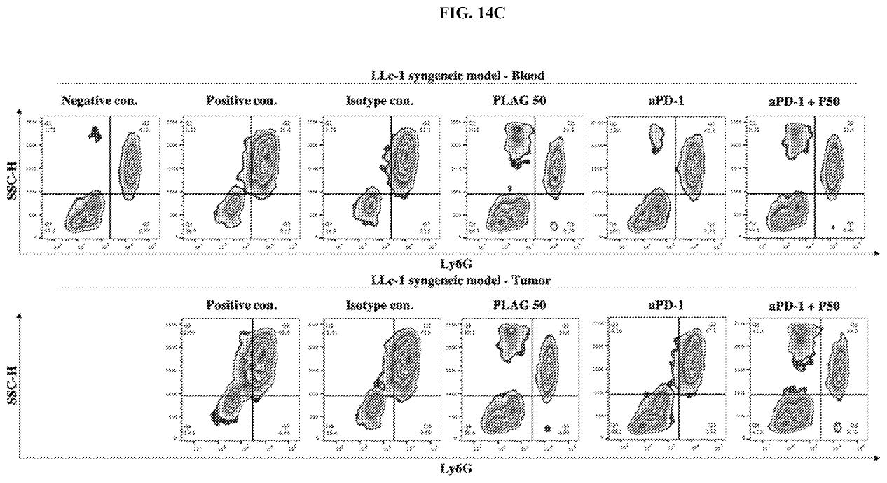

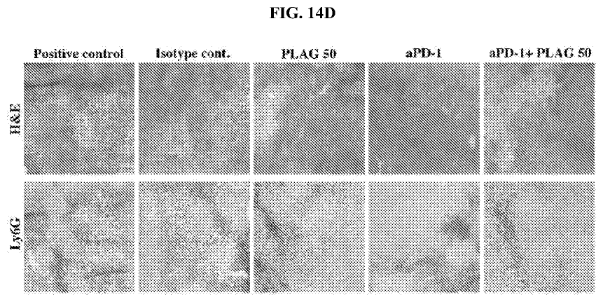

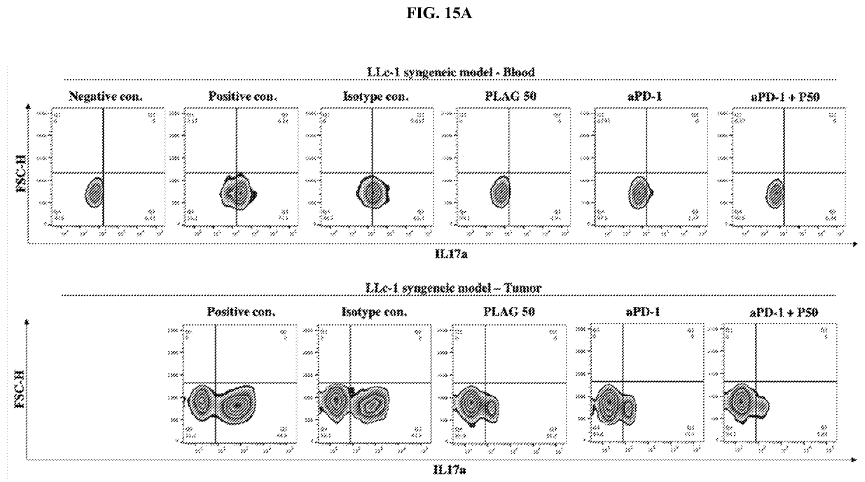

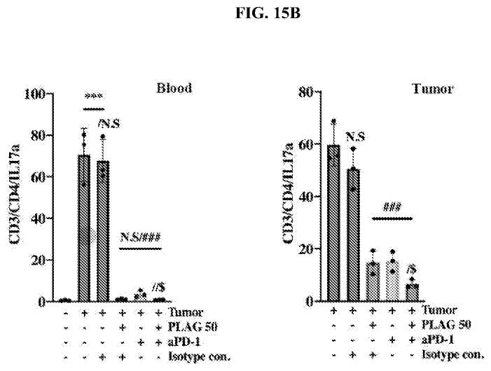

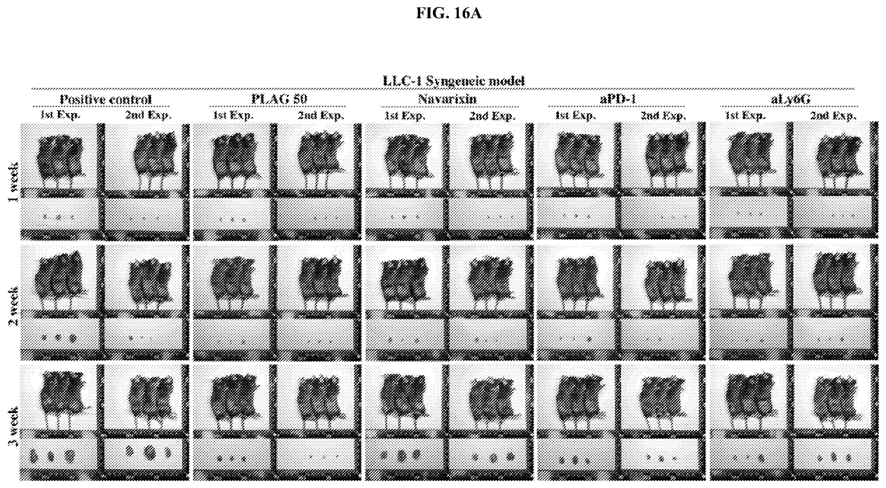

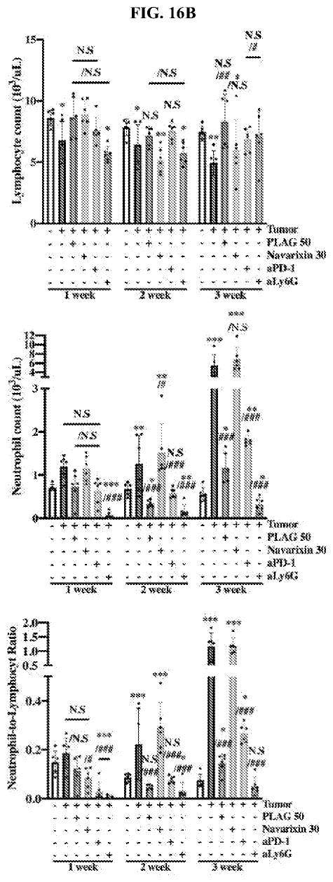

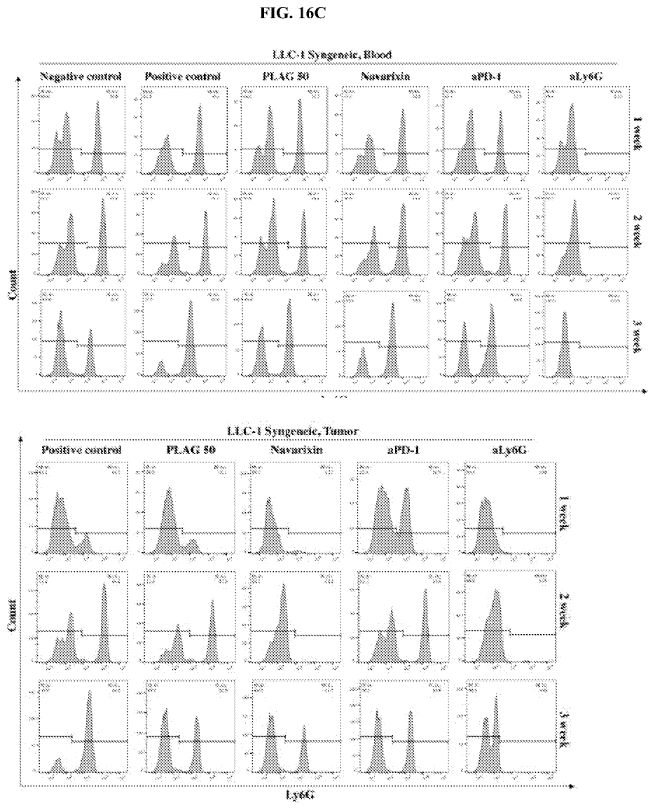

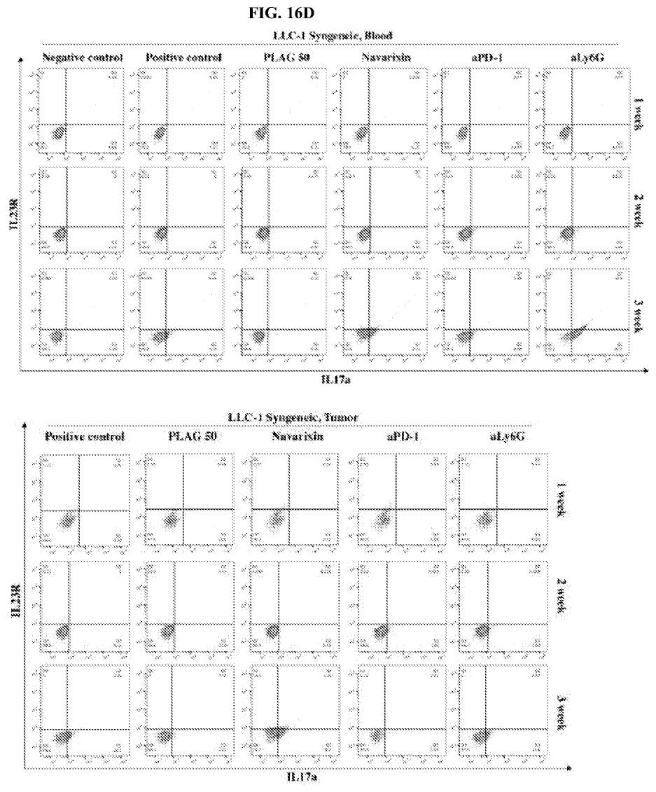

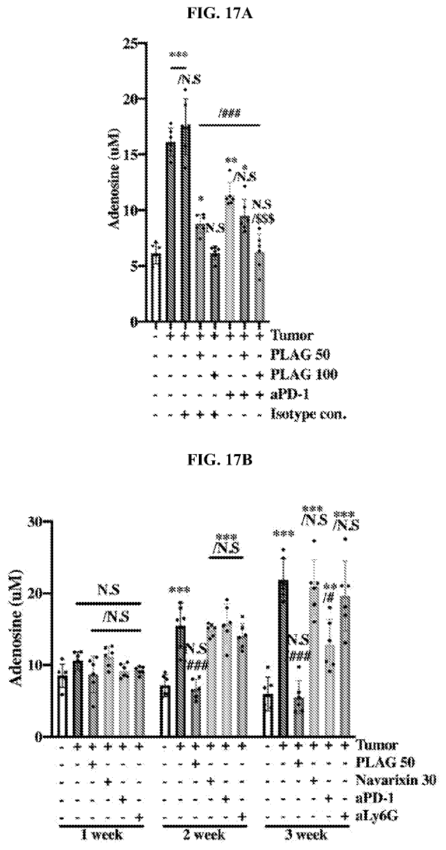

The neutrophil to lymphocyte ratio levels in the group co treated with PLAG were decreased remarkably compared to the aPD-1 alone. In particular, the degree of neutrophil infiltration in the tumor was effectively reduced upon PLAG treatment. Effects on the immune cell population and tumor infiltration by PLAG and aPD 1 treatment are demonstrated in FIGS. 14A-14D and effects on the modulation of Th17 population and tumor infiltration by PLAG and aPD-1 treatment are demonstrated in FIGS. 15A-15B. As shown in FIGS. 16A-16D, PLAG may act as a modulator, not an inhibitor of neutrophil infiltration and migration. Further as shown in FIGS. 17A-17B, PLAG may prevent the increase of DAMP by tumor progression through the rapid removal of DAMP.

The activity and infiltration of cytotoxic T-Lymphocyte (CTLs) in the tumor were effectively increased in the group co treated with PLAG compared to the aPD-1 alone. Such improvement was caused by a significant reduction of the population of Th 17 which induced massive neutrophil infiltration in the tumor, compared to the positive control.

### Conclusion

PLAG enhanced the anti cancer effect of aPD-1 synergistically on the regression of tumor burden via decreasing the tumor infiltrating neutrophils and Th 17 population while increasing the CTLs Therefore, combining aPD-1 with PLAG, which has excellent safety profiles, may contribute to enhancing the antitumor response of aPD-1 while lowering immune related toxicities by reducing the dose of ICI.

PLAG has not only a synergistic anti tumor effects on the tumor progression with aPD-1 but it suppress tumor progression on its own. PLAG reduced tumor infiltrating neutrophils (TIN) via an rapid removal of DAMP (adenosine) originated from tumor. By removal of the initial DAMP (adenosime) by PLAG, the massive infiltration of neutrophils to the tumor region is not occurred. PLAG reduced the Th 17 population and tumor infiltrating Th 17 cells involved in excessive neutrophil infiltration into tumor site.

Accordingly, combination of aPD-1 and PLAG may improve treatment outcomes of aPD-1 compared to aPD-1 alone contributing to enhancing anti tumor immune responses via treating the suppressive tumor microenvironment (TME). Presumably, PLAG treatment may transform the immunosuppressive TME into an immune enhanced TME via inhibition of neutrophil recruitment into the TME and enhancement of anti tumor immunity of T cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for treating a subject suffering from cancer, comprising:
administering to the subject:
a) one or more chemotherapeutic agents; and
b) a monoacetyl diacylglycerol compound of Formula (I): 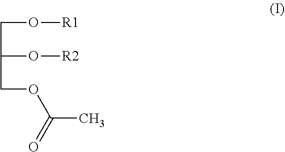
wherein R1 and R2 are independently a fatty acid group comprising 14 to 20 carbon atoms, wherein the compound of Formula (I) is distinct from the a) one or more chemotherapeutic agents.

2. The method of claim 1 wherein the a) one or more chemotherapeutic agents comprise an immune checkpoint inhibitor agent.

3. The method of claim 2 wherein the one or more immune checkpoint inhibitor agents comprise Pembrolizumab, Nivolumab, Cemiplimab, Atezolizumab, Avelumab, Durvalumab and/or Ipilmumab.

4. The method of claim 2 wherein the one or more immune checkpoint inhibitors include a PD-L1 inhibitor.

5. The method of claim 1 wherein the a) one or more chemotherapeutic agents comprise doxorubicin.

6. The method of claim 1 wherein the a) one or more chemotherapeutic agents comprise cyclophosphamide.

7. The method of claim 1 wherein the a) one or more chemotherapeutic agents comprise AC-regimen.

8. The method of claim 1 wherein the a) one or more chemotherapeutic agents is 5-FU, cisplatin, etoposide, ifosfamide, mesna, gemcitabine and/or tamoxifen.

9. The method of claim 1 wherein the monoacetyl diacylglycerol compound is a compound of Formula II:

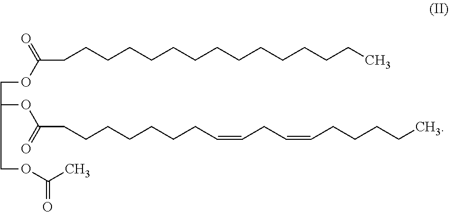 [SMILES: CCCCC/C=C\C/C=C\CCCCCCCC(=O)OC(COC(C)=O)COC(=O)CCCCCCCCCCCCCCC]

10-13. (canceled)

14. A method for treating a subject suffering from breast cancer, comprising:
administering to the subject:
a) doxorubicin and/or cyclophosamide; and
b) a monoacetyl diacylglycerol compound of Formula (I): 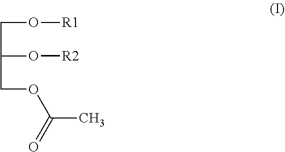
wherein R1 and R2 are independently a fatty acid group comprising 14 to 20 carbon atoms, wherein the compound of Formula (I) is distinct from the one or more chemotherapeutic agents.

15. The method of claim 14 wherein the subject is administered AC-regimen.

16. The method of claim 14 wherein the monoacetyl diacylglycerol compound is a compound of Formula II:

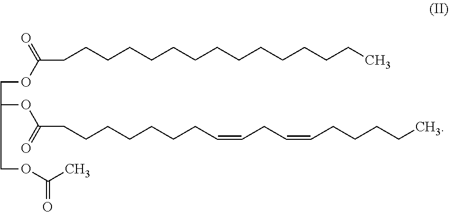 [SMILES: CCCCC/C=C\C/C=C\CCCCCCCC(=O)OC(COC(C)=O)COC(=O)CCCCCCCCCCCCCCC]

17. The method of claim 14 wherein the a) doxorubicin and/or cyclophosamide and b) compound of Formula (I) are administered substantially simultaneously.

18. The method of claim 14 wherein the a) doxorubicin and/or cyclophosamide and b) compound of Formula (I) are administered administered sequentially.

19. (canceled)

20. The method of claim 1 wherein the subject is a human.

21. (canceled)

22. A kit for the treatment of cancer comprising:
a) one or more chemotherapeutic agents; and
b) a monoacetyl diacylglycerol compound of Formula (I): 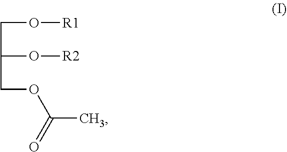
wherein R1 and R2 are independently a fatty acid group comprising 14 to 20 carbon atoms, and the compound of Formula (I) is distinct from the a) one or more chemotherapeutic agents.

23. The kit of claim 22 wherein the a) one or more chemotherapeutic agents comprise an immune checkpoint inhibitor agent.

24. The kit of claim 23 wherein the one or more immune checkpoint inhibitor agents comprise Pembrolizumab, Nivolumab, Cemiplimab, Atezolizumab, Avelumab, Durvalumab and/or Ipilmumab.

25. (canceled)

26. A kit for the treatment of cancer comprising:
a) doxorubicin and/or cyclophosamide; and
b) a monoacetyl diacylglycerol compound of Formula (I): 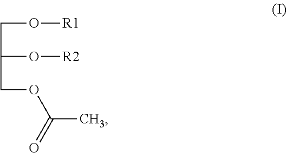
wherein R1 and R2 are independently a fatty acid group comprising 14 to 20 carbon atoms, and the compound of Formula (I) is distinct from the a) one or more chemotherapeutic agents.

27-29. (canceled)
